(19) United States
(12) Patent Application Publication
(10) Pub. No.: US 2025/0391007 A1
(43) Pub. Date: Dec. 25, 2025

(54) PREDICTION DEVICE, PREDICTION SYSTEM, AND PREDICTION PROGRAM

(71) Applicant: KONICA MINOLTA, INC., Chiyoda-ku, Tokyo (JP)

(72) Inventors: Miyuki OKANIWA, Tokyo (JP); Hiroshi KITA, Tokyo (JP); Osamu TOYAMA, Tokyo (JP); Yusuke KAWAHARA, Tokyo (JP); Kunimasa HIYAMA, Tokyo (JP)

(73) Assignee: KONICA MINOLTA, INC., Chiyoda-ku, Tokyo (JP)

(21) Appl. No.: 18/879,510

(22) PCT Filed: Jun. 28, 2023
(86) PCT No.: PCT/JP2023/023969

§ 371 Date: Dec. 27, 2024

(30) Foreign Application Priority Data
Jun. 30, 2022 (JP) 2022-105570

(51) Int. Cl. G06T 7/00; G06V 10/40; G06V 40/20

(52) U.S. Cl. CPC G06T 7/0004; G06V 10/40; G06V 40/20; G06T 2207/10048; G06T 2207/10056; G06T 2207/10064; G06T 2207/10116; G06T 2207/10132; G06T 2207/20081

(57) ABSTRACT

A prediction device includes an acquirer that acquires first information including an image regarding an object and second information including at least one of a character, a number, a chemical structure, and a spectrum regarding the object, and a predictor that predicts a plurality of characteristics of the object based on the acquired first information and the acquired second information.

### TECHNICAL FIELD

The present invention relates to a prediction device, a prediction system, and a prediction program.

### BACKGROUND ART

It is desired to promote digital transformation (DX) in various fields such as manufacturing industry, processing industry, and quality assurance, inspection, and analysis relating to or associated with the manufacturing industry and the processing industry. For example, methods for simplifying a step of inspecting the quality, physical properties, and the like of an object by using an image have been proposed (e.g., Patent Literature 1, Patent Literature 2, and the like).

By the way, it is not sufficient for a socially valuable product to satisfy a criterion for one characteristic such as one quality item or one physical property item, but it is desired to satisfy each criterion for a plurality of characteristics.

### CITATION LIST

### Patent Literature

Patent Literature 1: JP 2019-184450 A
Patent Literature 2: JP 2014-193596 A

### SUMMARY OF INVENTION

### Technical Problem

Therefore, it is desirable to be able to concurrently predict a plurality of characteristics of an object.

The present invention has been made in view of the above-described circumstances, and an object of the present invention is to provide a prediction device, a prediction system, and a prediction program that are capable of predicting a plurality of characteristics of an object.

### Solution to Problem

The above-described object of the present invention is achieved by the following means.

(1) A prediction device including: an acquirer that acquires first information including an image regarding an object and second information including at least one of a character, a number, a chemical structure, and a spectrum regarding the object; and a predictor that predicts a plurality of characteristics of the object based on the acquired first information and the acquired second information.

(2) The prediction device according to (1), further comprising a selector that selects the first information and the second information in accordance with the plurality of characteristics of the object to be predicted, wherein the predictor predicts the plurality of characteristics of the object based on the selected first information and the selected second information.

(3) The prediction device according to (1), wherein the image includes an image obtained by imaging the object using at least one of an imaging device, an X-ray Talbot-Lau device, an ultrasonic device, a fluorescent fingerprint measurement device, a hyperspectral camera, a millimeter wave imaging device, a scanning electron microscope, an atomic force microscope, a transmission electron microscope, a fluorescence microscope, and a multidimensional colorimeter.

(4) The prediction device according to (1), wherein the image includes an image obtained by imaging a behavior of a person related to the object.

(5) The prediction device according to (1), wherein the second information includes at least one of a character and a chemical structure representing a type of a substance contained in the object, and a number representing an amount of the substance contained in the object.

(6) The prediction device according to (1), wherein the second information includes at least one of an infrared absorption spectrum, a terahertz wave spectroscopy spectrum, a nuclear magnetic resonance spectrum, a Raman spectroscopy spectrum, an impedance spectroscopy spectrum, and an X-ray diffraction spectrum of the object.

(7) The prediction device according to (1), wherein the object is a mixture of a plurality of substances having chemical structures different from each other.

(8) The prediction device according to (1), wherein the plurality of characteristics include at least one of a physical property, quality, and a function of the object.

(9) The prediction device according to (1), wherein the plurality of characteristics include at least one of a mechanical property, a physical property, a thermal characteristic, moldability, an electrical characteristic, durability, machinability, and combustibility of the object.

(10) The prediction device according to (1), further including a controller that causes an output section to output information regarding the plurality of predicted characteristics.

(11) The prediction device according to (1), wherein the predictor predicts the plurality of characteristics using a trained discriminator.

(12) The prediction device according to (11), further including an extractor that extracts a feature from each of the acquired first information and the acquired second information, wherein the predictor predicts the plurality of characteristics with the extracted features as inputs.

(13) The prediction device according to (12), wherein the discriminator is subjected to machine learning with the features as input data and the plurality of characteristics as output data.

(14) A prediction system including: a first device that generates first information regarding an object; a second device that generates second information regarding the object; and the prediction device according to any one of (1) to (13).

(15) A prediction program for causing a computer to execute a process including: (a) acquiring first information including an image regarding an object and second information including at least one of a character, a number, a chemical structure, and a spectrum regarding the object; and (b) predicting a plurality of characteristics of the object based on the acquired first information and the acquired second information.

### Advantageous Effects of Invention

A prediction device, a prediction system, and a prediction program according to the present invention acquire first information regarding an object and second information regarding the object, and predict a plurality of characteristics of the object based on the acquired first information and the acquired second information. Thus, the plurality of characteristics of the object can be predicted concurrently.

### BRIEF DESCRIPTION OF DRAWINGS

FIG. 1 is a diagram illustrating an overall configuration of a prediction system according to an embodiment.

FIG. 2 is a block diagram illustrating a schematic configuration of a prediction device.

FIG. 3 is a diagram illustrating another example of the prediction system illustrated in FIG. 1.

FIG. 4 is a diagram illustrating still another example of the prediction system illustrated in FIG. 1.

FIG. 5 is a block diagram illustrating a functional configuration of the prediction device.

FIG. 6 is a diagram illustrating an example of a display format of information output by the prediction device.

FIG. 7 is a flowchart illustrating a procedure of prediction processing executed in the prediction device.

FIG. 8 is a flowchart illustrating a machine learning method for a trained model.

FIG. 9 is a block diagram illustrating a functional configuration of a prediction device according to a modification example.

FIG. 10 is a flowchart illustrating a procedure of prediction processing executed in the prediction device illustrated in FIG. 9.

### DESCRIPTION OF EMBODIMENTS

Hereinafter, embodiments of the present invention will be described with reference to the accompanying drawings. Note that in the description of the drawings, the same elements are denoted by the same reference signs, and redundant descriptions are omitted. In addition, dimensional ratios in the drawings are exaggerated for convenience of the description and may be different from actual ratios.

### EMBODIMENTS

### <Configuration of Prediction System>

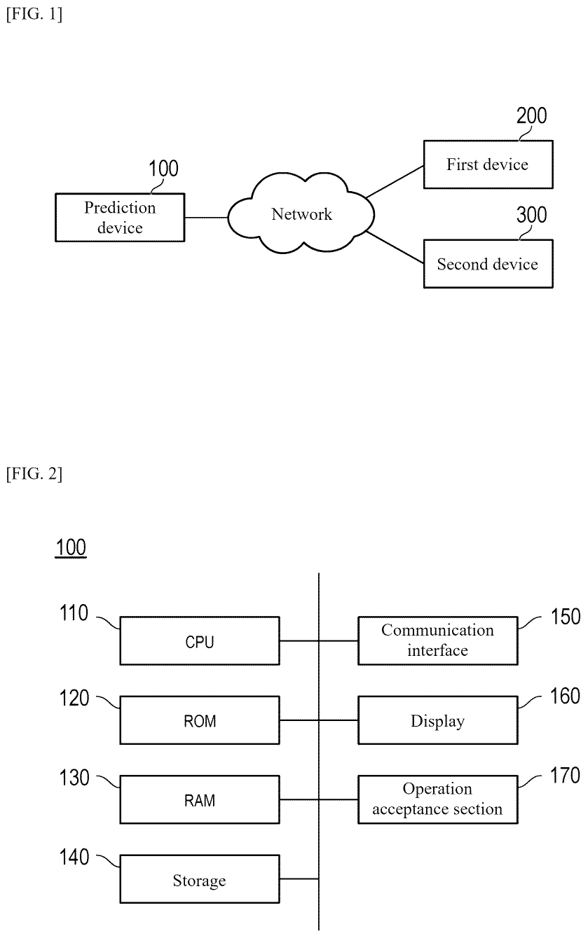

FIG. 1 is a diagram illustrating an overall configuration of a prediction system.

As illustrated in FIG. 1, the prediction system includes, for example, a prediction device 100, a first device 200, and a second device 300. The prediction system predicts a plurality of characteristics of an object using scientific information and non-scientific information regarding the object. In this embodiment, the non-scientific information corresponds to a specific example of first information in the present invention, and the scientific information corresponds to a specific example of second information in the present invention.

Examples of the object include a fiber composite material and fiber-reinforced plastics (FRPs). The FRPs are composite materials in which carbon fiber, glass fiber, cellulose fiber, cellulose nanofiber, or the like is used as reinforced fiber. The FRPs include, for example, carbon-fiber-reinforced plastics (CFRPs), carbon fiber reinforced thermoplastics (CFRTPs), glass-fiber-reinforced plastics (GFRPs), cellulose-fiber-reinforced plastics (CeFRPs), and the like. Fiber composite materials and FRPs are used as constituent members of various products and the like. The products are, for example, space and aircraft related products, automobiles, ships, fishing rods, electric, electronic, and household electric appliance components, parabolic antennas, bathtubs, floor materials, roof materials, and the like. In particular, CFRTPs are excellent in terms of lightweight and recyclability.

The object may be a material other than a composite material using resin as a matrix as described above. The object may be, for example, a composite material such as a rubber matrix composite (RMC) using rubber, a metal matrix composite (MMC) using metal, a ceramics matrix composite (CMC) using a ceramic, or the like. The object may be an industry product such as concrete or asphalt, a food product, or the like.

Specifically, the object is, for example, a mixture of a plurality of substances having chemical structures different from each other. The object is, for example, a composite material containing a filler and a resin. The resin contained in the composite material is, for example, a known thermosetting resin, a known thermoplastic resin, or the like. Specific examples of the resin include polyolefin resin such as polyethylene resin (PE), polypropylene resin (PP), and maleic anhydride-modified polypropylene (MAHPP), epoxy resin, phenol resin, unsaturated polyester resin, vinyl ester resin, polycarbonate resin, polyester resin, polyamide (PA) resin, liquid crystal polymer resin, polyether sulfone resin, polyetheretherketone resin, polyarylate resin, polyphenylene ether resin, polyphenylene sulfide (PPS) resin, polyacetal resin, polysulfone resin, polyimide resin, polyetherimide resin, polystyrene resin, modified polystyrene resin, AS resin (copolymer of acrylonitrile and styrene), ABS resin (copolymer of acrylonitrile, butadiene, and styrene), modified ABS resin, MBS resin (copolymer of methyl methacrylate, butadiene, and styrene), modified MBS resin, polymethyl methacrylate (PMMA) resin, modified polymethyl methacrylate resin, and the like. The resin contained in the composite material may be one of these, or two or more of these may be mixed.

The filler contained in the composite material is added to the resin, for example, for the purpose of improving the strength of the composite material. The filler is added to the resin at a concentration of, for example, 0.1% to 50% by volume. The filler has, for example, a fiber shape or a particle shape. Examples of the fiber-shaped filler include glass fiber (GF), carbon fiber (CF), aramid fiber, alumina fiber, silicon carbide fiber, boron fiber, silicon carbide fiber, and the like. For the CF, for example, polyacrylonitrile (PAN-based), pitch-based, cellulose-based, or hydrocarbon vapor-grown carbon fiber, and graphite fiber may be used. In addition, for the GF, for example, E glass, S glass, and the like may be used. The composite material preferably contains at least one of glass fiber (GF) and carbon fiber (CF). Since the orientation state of the filler in the composite resin containing at least one of glass fiber (GF) and carbon fiber is easily measured by an X-ray Talbot-Lau device described later, it is possible to improve the accuracy of predicting a plurality of characteristics.

The particle-shaped filler is, for example, inorganic particles such as a calcium carbonate (CaCo₃), talc (Mg₃Si₄O₁₀(OH)₂), barium sulfate (BaSO₄), mica (Si, Al, Mg, K), aluminum hydroxide (Al(OH)₃), magnesium hydroxide (Mg(OH)₂), titanium oxide (TiO₂), zinc oxide (ZnO₂), antimony oxide (Sb₂O₃), kaolinic clay (Al₂O₃·2SiO₂·2H₂O), and carbon black. The filler contained in the object may be one of these, or two or more of these may be mixed.

The composite material may contain a sensitivity adjuster. The sensitivity adjuster refers to a material that functions like an iodine-based contrast agent used in medical CT imaging. For example, in a case where the composite material contains the sensitivity adjuster, an image with higher contrast can be formed. Alternatively, in a case where the composite material contains the sensitivity adjuster, a phenomenon serving as a feature is highlighted, or a phenomenon serving as a feature can be detected, and thus the feature is easily grasped. The sensitivity adjuster is preferably used at the time of acquiring the non-scientific information. For example, in a case where the second device 300 is a Raman spectrometer, when zirconium tungstate is used as the sensitivity adjuster, a Raman shift changes and it is possible to generate information regarding the material characteristics of the fiber composite material with higher accuracy. For example, in a case where the first device 200 is a fluorescence microscope, when a fluorescent dye is used as the sensitivity adjuster, it is possible to generate information regarding the length of the fiber with higher accuracy.

The sensitivity adjuster contained in the composite material preferably has a small effect on the physical properties of the composite material. Thus, for example, the composite material measured by the first device 200 and the second device 300 can be used for, for example, a molded product or the like. For measurement with the first device 200 and the second device 300, a test piece of the composite material containing the sensitivity adjuster may be prepared. The sensitivity adjuster is appropriately selected, for example, in accordance with the composite material or in accordance with the characteristics of the composite material. As the sensitivity adjuster, for example, a dye is used. Examples of the dye include a fluorescent dye, a heat-sensitive dye, and a pressure-sensitive dye. An additive added to the composite material for a purpose other than sensitivity adjustment may function as a sensitivity adjuster. Examples of the additive include a plasticizer, an antioxidant, an ultraviolet absorber, a nucleating agent, a transparentizing agent, a flame retardant, and the like.

The object may be an alloy, fiber, ceramics, paper, a synthetic resin, a liquid crystal polymer, a cultured cell, a biomaterial, or the like. The biomaterial is, for example, a bone, a cell, or blood.

### (Prediction Device 100)

The prediction device 100 is, for example, a computer such as a personal computer (PC), a smartphone, or a tablet terminal and functions as a prediction device in the present embodiment. The prediction device 100 is configured to be connectable to the first device 200 and the second device 300, and transmits and receives various types of information to and from each of the devices.

FIG. 2 is a block diagram illustrating a schematic configuration of the information processing apparatus.

As illustrated in FIG. 2, the prediction device 100 includes a central processing unit (CPU) 110, a read only memory (ROM) 120, a random access memory (RAM) 130, a storage 140, a communication interface 150, a display 160, and an operation acceptance section 170. The components are communicably connected to each other via a bus.

The CPU 110 controls the above-described components and performs various types of arithmetic processing in accordance with a program recorded in the ROM 120 or the storage 140.

The ROM 120 stores various types of programs or various types of data.

The RAM 130, as a workspace, temporarily stores a program and data.

The storage 140 stores various programs including an operating system or various types of data. For example, an application for predicting the plurality of characteristics of the object from the non-scientific information and the scientific information, which will be described later, using a trained discriminator is installed in the storage 140. Further, the storage 140 may store the non-scientific information and the scientific information acquired from the first apparatus 200 and the second apparatus 300. Further, in the storage 140, a trained model to be used as the discriminator or teacher data to be used for machine learning may be stored.

The communication interface 150 is an interface for communicating with the other devices. As the communication interface 150, a communication interface based on various wired or wireless standards is used. The communication interface 150 is used, for example, in order to receive the non-scientific information and the scientific information from the first device 200 or the second device 300, or in order to transmit a result of predicting the plurality of characteristics to another device such as a server for storage.

The display 160 includes a liquid crystal display (LCD), an organic EL display, or the like, and displays various types of information. The display 160 may be configured by viewer software, a printer, or the like. In the present embodiment, the display 160 functions as an output section.

The operation acceptance section 170 includes a touch sensor, a pointing device such as a mouse, a keyboard, or the like, and accepts various user operations. The display 160 and the operation acceptance section 170 may form a touch screen by superimposing a touch sensor as the operation acceptance section 170 on a display surface as the display 160.

### (First Device 200)

The first device 200 is a device for generating the non-scientific information regarding the object. In this case, the non-scientific information is information obtained by processing data acquired for analyzing, analyzing, or evaluating the performance, function, quality, or the like of the predetermined target. This processing will be described taking as an example a case where the first device 200 is an imaging device such as a digital camera.

In the digital camera, light that enters from a lens is imaged on an image sensor, and the sensor detects the light and converts the light into digital data. An image of a digital camera photograph is generated by processing this data with an image processing engine. For example, in the case of an image with one million pixels, the digital camera processes, with the image processing engine, a plurality of pieces of information sensed by one million image sensors, that is, multidimensional data, to reconstruct the image into a two-dimensional image. The plurality of pieces of information are, for example, information such as intensities of RGB. Since such an image originally includes multidimensional data, it is possible to obtain new information that cannot be obtained from the scientific information.

The non-scientific information includes, for example, an image regarding the object. The image may be either a moving image or a still image. The image may be an image such as a moving image obtained by imaging a behavior of a person related to the object. The person related to the object is, for example, a person involved in the manufacturing of the object. In the manufacturing of the composite material, not only an automated step using a robot or the like but also a step involving a human manipulation may be present. In particular, at the time of development of the composite material, it frequently occurs that a manufacturing process, measurement content, and the like vary depending on a target, a phase, and the like. Therefore, it is difficult to automate all steps, and a step involving a manipulation is often present. For example, a moving image of a step involving a manipulation is captured using the first device 200 such as a video camera. From the captured image, the prediction device 100 detects a person and his/her motion using, for example, OpenPose or the like, and extracts a specific motion. The prediction device 100 obtains, for example, an agent input speed, an agent input timing, an agent input interval, a stirring speed, a stirring time, or the like from the extracted motion, and uses these as features for characteristic prediction. The prediction device 100 may use machine learning for the extraction of the specific motion and the extraction of the features. In this case, the image itself captured by the first device 200 is not classified into the scientific information because the information included in the image varies depending on a manipulation for which the image is captured. Note that the features extracted from the image can be scientific information. For example, a feature determined according to the target or the manipulation content is extracted from the image. The imaging device may be, for example, the above-described digital camera or the like, or may be MOBOTIX (registered trademark) or the like.

The first device 200 is a device that generates such non-scientific information. The first device 200 includes a device that generates an image of the object, for example, at least one of an imaging device, an X-ray Talbot-Lau device, an ultrasonic device, a fluorescent fingerprint measurement device, a hyperspectral camera, a millimeter wave imaging device, a scanning electron microscope, an atomic force microscope, a fluorescence microscope, and a multidimensional colorimeter.

### (Second Device 300)

The second device 300 is a device for generating the scientific information regarding the object. In this case, the scientific information is information to be contrasted with the non-scientific information described above. The scientific information is information itself detected by a sensor, that is, information that has not been processed to be multidimensional. The scientific information may be information before multi-dimensionalization processing, that is, so-called raw data. For example, the second device 300 is a light receiving element (or a light receiving pixel) or the like of an imaging device, and information (digital data) detected by the light receiving element is the scientific information.

The scientific information is primary information from which a phenomenon occurring in the object is directly grasped. This scientific information tends to be directly associated with the mechanism of a reaction occurring in the object and a mechanism by which a function of the object is expressed. The scientific information in this case is one-dimensional information, and includes, for example, at least one of a character, a number, a chemical structure, and a spectrum regarding the object.

For example, the scientific information includes at least one of a character, a number, a chemical structure, and a spectrum representing a substance (hereinafter referred to as a contained substance) contained in the object. Specifically, the scientific information includes at least one of a character and a chemical structure representing the type of the contained substance, and a number representing the amount of the contained substance. The contained substance may be a main component or may be an impurity. The scientific information may include a number representing the purity of at least one of the object and the contained substance. The scientific information may include a character representing a form of at least one of the object and the contained substance. The form is, for example, solid, liquid, gel, or the like.

For example, the scientific information includes at least one of a character and a number representing a condition for manufacturing the object. Specifically, the scientific information includes at least one of a character and a number representing the temperature, time, content, pressure, speed, or the like of each process of manufacturing the object.

For example, the scientific information includes a signal value or the like to be used for the analysis and the like of the object. This signal value may have undergone processing other than multi-dimensionalization. The processing other than the multi-dimensionalization is, for example, processing such as addition, subtraction, multiplication, division, and ratio change.

For example, the scientific information may include a spectrum of the object or the like. The spectrum of the object includes, for example, at least one of an infrared absorption spectrum, a terahertz wave spectroscopy spectrum, a nuclear magnetic resonance spectrum, a Raman spectroscopy spectrum, an impedance spectroscopy spectrum, and an X-ray diffraction spectrum.

Since the spectrum is not information as an integrated image, the spectrum is not classified into the non-scientific information. Since the spectrum is a set of one-dimensional information of each point, the spectrum corresponds to scientific information. The one-dimensional information of each point is, for example, infrared absorption intensity at a predetermined wavenumber, or the like.

The spectrum includes a one-dimensional spectrum and a multidimensional spectrum having two or more dimensions, and a two-dimensional spectrum is referred to as imaging in some cases. When not identified, the spectrum means a one-dimensional spectrum, but the one-dimensional spectrum is scientific information and the multidimensional spectrum is non-scientific information.

As an example of the one-dimensional spectrum and the multidimensional spectrum, NMR will be described. A one-dimensional NMR spectrum includes, for example, a proton (1H) and carbon (13C). In 1H-NMR, information such as the structure of C where His present, the presence of adjacent nuclei, and the number of H atoms can be obtained from the chemical shift, the spin-spin coupling, and the integral value. The structure of C where His present is, for example, H bonded to primary carbon and the like. That is, 1H-NMR represents, as information about the vicinity of the presence of specific H, information about the characteristics of carbon bonded, the number of H atoms in the same environment, and the like. With this information, it may be possible to identify the structure in a case where the molecular structure can be estimated to some extent, but with this information alone, information about only a part of the molecule, such as the number of H atoms, can be obtained. A two-dimensional NMR spectrum is a measurement method in which a correlation between signals or a spin division pattern of each signal is two-dimensionally developed with frequencies as a vertical axis and a horizontal axis, and the intensity of its peak is displayed by using a contour diagram or the like. Examples of the two-dimensional NMR spectrum include COSY and CHCOSY. In particular, this two-dimensional NMR spectrum is utilized in a case where the object has a complex chemical structure. Since CHCOSY is heteronuclear shift correlation two-dimensional NMR, it is possible to identify which C and H are bonded. That is, it can be said that it is possible to identify the entire molecular structure with non-scientific information, and it can be said that new information which cannot be obtained only with scientific information which is one-dimensional NMR can be obtained.

The second device 300 is a device that generates such scientific information. The second device 300 includes, for example, a light receiving element of an imaging device or the like. The second device 300 may include a luminescent DNA sensor or the like. The second device 300 may include a computer or the like to which at least one of a character, a number, a chemical structure, and a spectrum representing the contained substance is input. Alternatively, the second apparatus 300 may include a computer, a sensor, or the like to which at least one of a character and a numerical value representing a condition for manufacturing the object is input. The second device 300 may include a device that performs analysis or the like of the object. Alternatively, the second device 300 may include at least one of an infrared spectrometer, a terahertz wave spectrometer, a nuclear magnetic resonance device, a Raman spectrometer, an impedance spectrometer, or an X-ray diffraction device that generates each spectrum of the object.

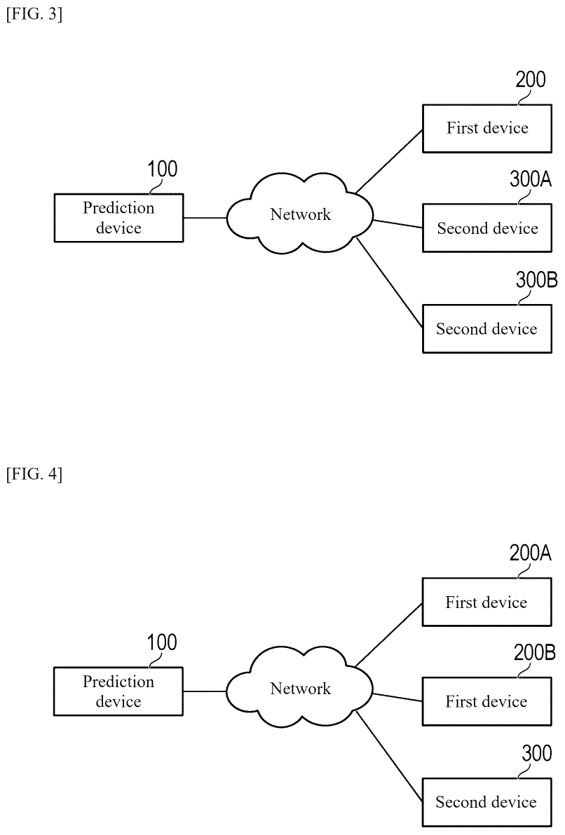

FIGS. 3 and 4 illustrate other examples of the prediction system. The prediction system may include a plurality of first devices 200 (e.g., first devices 200A and 200B illustrated in FIG. 3) and may include a plurality of second devices 300 (e.g., second devices 300A and 300B illustrated in FIG. 4). The prediction system may include a plurality of first devices 200 and a plurality of second devices 300 (not illustrated).

### <Functions of Prediction Device 100>

FIG. 5 is a block diagram illustrating a functional configuration of the prediction device 100.

As illustrated in FIG. 5, the prediction device 100 functions as an acquirer 111, an extractor 112, a predictor 113, and a controller 114 when a CPU 110 reads a program stored in the storage 140 and executes processing.

The acquirer 111 acquires the non-scientific information generated by the first device 200 and the scientific information generated by the second device 300. The non-scientific information includes, for example, an image regarding the object, and the scientific information includes, for example, at least one of a character, a number, a chemical structure, and a spectrum regarding the object. The acquirer 111 preferably acquires a plurality of pieces of scientific information and a plurality of pieces of non-scientific information. Accordingly, it is possible to predict the characteristics of the object with higher accuracy.

The extractor 112 extracts a feature from each of the non-scientific information and the scientific information acquired by the acquirer 111. The extractor 112 may extract a plurality of features from each of the non-scientific information and the scientific information.

The acquirer 111 may acquire information from which the features have been extracted. That is, the non-scientific information and the scientific information may be information in which the features are extracted from the information regarding the object generated by the first device 200 and the second device 300.

The predictor 113 predicts a plurality of characteristics of the object based on the non-scientific information and the scientific information acquired by the acquirer 111. Specifically, the predictor 113 predicts, by using the trained discriminator, the plurality of characteristics of the object with the features of the non-scientific information and the scientific information extracted by the extractor 112 as inputs.

The characteristics of the object include, for example, at least one of physical properties, quality, and functions of the object. Specifically, the physical properties of the object include at least one of mechanical properties, physical properties, thermal characteristics, moldability, electrical characteristics, and durability of the object. The mechanical properties of the object include, for example, mechanical strength, elastic modulus, bending strength, bending elastic modulus, impact strength, hardness, and the like of the object. The physical properties of the object include, for example, the density and the like of the object. The thermal characteristics of the object include, for example, the thermal conductivity, specific heat, thermal expansion coefficient, and deflection density under load of the object. The moldability of the object includes, for example, the compression molding temperature, injection molding temperature, solution viscosity, molding shrinkage rate, and the like of the object. The electrical characteristics of the object include, for example, the volume resistance, insulation breaking strength, dielectric constant, and arc resistance of the object. The durability of the object includes, for example, the weak acid resistance, strong acid resistance, weak base resistance, strong base resistance, organic solvent resistance, light resistance, weather resistance, and the like of the object. The physical properties of the object may include machinability, combustibility, and the like.

For example, according to ISO9000, the quality of the object refers to the extent to which a collection of characteristics (3.10.1) inherent in the object (3.6.1) satisfies the requirements (3.6.4). For example, the quality of a component used in a car refers to the exterior related to appearance, lightweight related to a running distance and fuel efficiency, the durability of a component related to the life of the car, and the like. When the focus is placed on the manufacturing thereof, the following are exemplified: the number of components is reduced, a plurality of functions are included in one component, the easiness of processing and manufacturing, energy-saving manufacturing with no environmental load, recyclability, and the like. Functions of the target include, for example, impact absorption, plasticity, transparency, flame retardancy, and antistatic and slip properties.

The predictor 113 preferably predicts a plurality of characteristics different from each other. For example, the predictor 113 predicts a plurality of characteristics different from each other among mechanical properties, physical properties, thermal characteristics, moldability, electrical characteristics, durability, machinability, combustibility, and the like of the object. The predictor 113 predicts, for example, mechanical properties including the mechanical strength and the impact strength, and moldability including the molding shrinkage rate.

The predictor 113 determines a plurality of characteristics to be predicted, based on, for example, an instruction input in advance from a user. The user inputs the instruction, for example, via the operation acceptance section 170. The predictor 113 may determine a plurality of predictable characteristics based on the scientific information and the non-scientific information regarding the object acquired by the acquirer 111.

The controller 114 causes the display 160 to output information regarding the plurality of characteristics of the object predicted by the predictor 113.

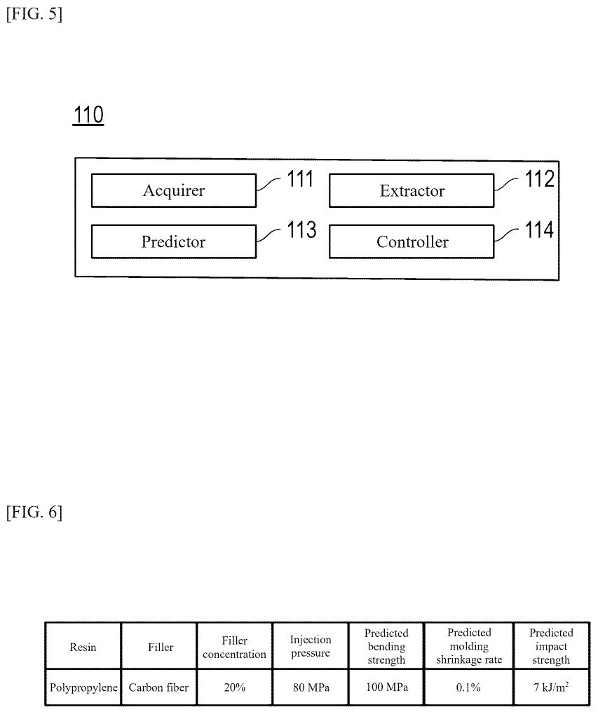

FIG. 6 illustrates an example of the information regarding the plurality of characteristics of the object output to the display 160. The display 160 displays, for example, values of the plurality of predicted characteristics together with the information regarding the object.

Processing executed in the prediction device 100 will be described in detail below.

### <Overview of Processing>

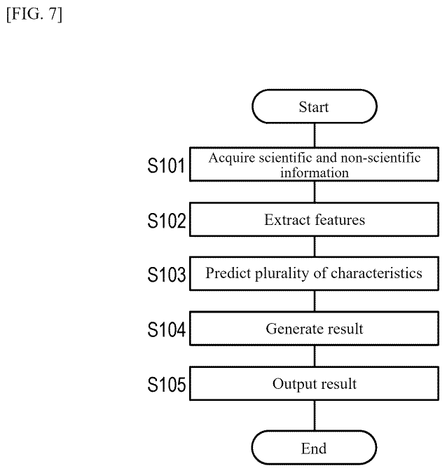

FIG. 7 is a flowchart illustrating a procedure of prediction processing executed in the prediction device 100. The processing of the prediction device 100 illustrated in the flowchart of FIG. 7 is stored as a program in the storage 140 of the prediction device 100, and is executed by the CPU 110 controlling each section.

### (Step S101)

First, the prediction device 100 acquires the non-scientific information regarding the object generated by the first device 200 and the scientific information regarding the object generated by the second device 300. For example, the prediction device 100 acquires the non-scientific information from the first device 200 and the scientific information from the second device 300. The first device 200 and the second device 300 may store the non-scientific information and the scientific information to another device such as a server, and the prediction device 100 may acquire the non-scientific information and the scientific information from the other device.

### (Step S102)

The prediction device 100 extracts the features from each of the non-scientific information and the scientific information acquired in the processing in step S101.

### (Step S103)

The prediction device 100 predicts the plurality of characteristics of the object by inputting the features of each of the non-scientific information and the scientific information extracted in the processing in step S102 to the discriminator that has been subjected to machine learning in advance. For example, the discriminator is subjected to machine learning using teacher data by a learning method to be described later. The teacher data includes features of each of non-scientific information and scientific information of a plurality of objects prepared in advance, and measured values of a plurality of characteristics of each of the plurality of objects.

Specifically, the discriminator is subjected to machine learning using the features extracted from the non-scientific information and the scientific information regarding the plurality of objects as input data and using the measured values of the plurality of characteristics of each of the plurality of objects as output data. Since the machine learning is performed for each characteristic, a feature group suitable for prediction of each characteristic is found. There is a known technique for automatically extracting a feature from non-scientific information including an image by deep learning. By using this technology, a pattern or a common point is found from an enormous amount of data, and a feature is extracted. Therefore, even in a case where a factor affecting a predetermined characteristic is not clear, that is, in a case where a mechanism by which the predetermined characteristic is expressed is not sufficiently understood, it is possible to extract a feature suitable for the prediction of the characteristic. Therefore, it is preferable to use deep learning for extraction of a feature of non-scientific information such as an image. Accordingly, the prediction device 100 can predict the plurality of characteristics of the object by inputting the features extracted for each of the non-scientific information and the scientific information to the discriminator.

The discriminator may be subjected to machine learning using the non-scientific information and scientific information regarding the plurality of objects as input data and using the measured values of the plurality of characteristics of each of the plurality of objects as output data. Further, the information to be input to the discriminator is not limited to the features of the non-scientific information and the scientific information regarding the object. For example, in addition to the features of each of the non-scientific information and the scientific information regarding the object, other information may be input to the discriminator and used as information for performing learning and prediction.

### (Step S104)

The prediction device 100 generates a result of predicting the plurality of characteristics of the object, based on the output from the discriminator in the processing in step S103.

### (Step S105)

The prediction device 100 outputs the prediction result generated in the processing in step S104. For example, the prediction device 100 displays, on the display 160, a value of each of the plurality of characteristics predicted in the processing in step S103 together with the information regarding the object (FIG. 6).

### <Regarding Learning Processing>

Next, a machine learning method for the trained model to be used in the discriminator will be described.

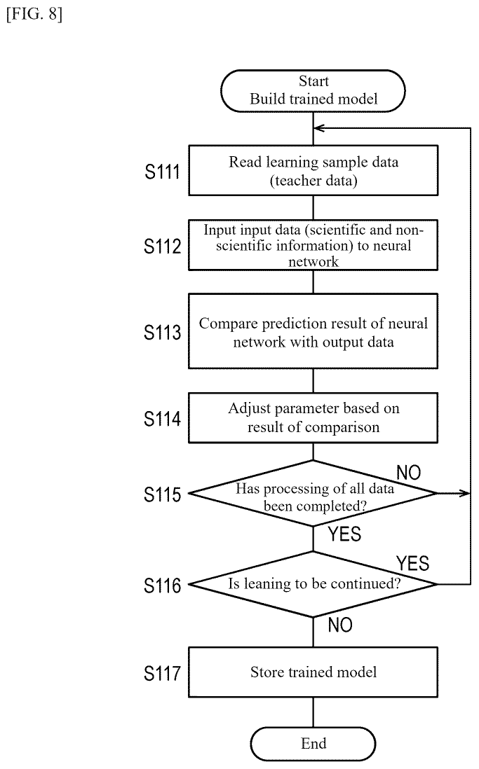

FIG. 8 is a flowchart illustrating the machine learning method for the trained model.

In processing illustrated in FIG. 8, machine learning is executed using a large number (i sets) of data sets prepared in advance as learning sample data. The data sets are data sets in which the features of each of the non-scientific information and the scientific information of the plurality of objects are used as inputs and the measured values of the plurality of characteristics of each of the plurality of objects are used as outputs. For example, a stand-alone high-performance computer using processors such as a CPU and a GPU or a cloud computer is used as a learning device (not illustrated) that functions as the discriminator. A learning method using a neural network formed by combining perceptrons, such as deep learning, in the learning device will be described below, but the present invention is not limited thereto, and various methods can be applied. For example, a random forest, a decision tree, a support vector machine (SVM), logistic regression, a k-nearest neighbor algorithm, a topic model, and the like can be applied.

### (Step S111)

The learning device reads the learning sample data that is teacher data. In a case where the reading is to be performed for the first time, the first set of learning sample data is read, and in a case where the reading is performed for the i-th time, the i-th set of learning sample data is read.

### (Step S112)

The learning device inputs input data among the read learning sample data to the neural network.

A pseudo image may be used for the non-scientific information and the scientific information that serve as the learning sample data. The pseudo image is an image created in a pseudo manner based on original data. In this case, the original data may be either the scientific information or the non-scientific information. When the original data is the scientific information, the pseudo image is treated as the scientific information, and when the original data is the non-scientific information, the pseudo image is treated as the non-scientific information.

The pseudo image is, for example, a pseudo image created in a pseudo manner as an image obtained by imaging the object using at least one of an imaging device, an X-ray Talbot-Lau device, an ultrasonic device, a fluorescent fingerprint measurement device, a hyperspectral camera, a millimeter wave imaging device, a scanning electron microscope, an atomic force microscope, a transmission electron microscope, a fluorescence microscope, and a multidimensional colorimeter. For example, a Talbot image (pseudo Talbot image) of the object may be generated in a pseudo manner by using, as the original data, a plurality of images obtained by imaging a composite material with a material and a mixing ratio similar to those of the object by an X-ray Talbot-Lau device.

### (Step S113)

The learning device compares a prediction result of the neural network with correct data.

### (Step S114)

The learning device adjusts a parameter based on a result of the comparison. The learning device adjusts the parameter so as to reduce a difference in the result of the comparison, for example, by executing processing based on backpropagation (back-propagation).

### (Step S115)

When the processing of all data of the first to i-th sets has been completed (YES), the learning device advances the processing to step S116. When the processing has not been completed (NO), the learning device returns the processing to step S111, reads the next learning sample, and repeats the processing from step S111.

### (Step S116)

The learning device determines whether or not to continue the learning. In a case where the learning is to be continued (YES), the learning device returns the processing to step S111 and executes the processing on the first to i-th sets again in steps S111 to S115. In a case where the learning is not to be continued (NO), the learning device advances the processing to step S117.

### (Step S117)

The learning device stores the trained model built by the processing so far, and ends the processing (end). A destination to which the trained model is stored includes an internal memory of the prediction device 100. In the processing illustrated in FIG. 7 described above, the plurality of characteristics of the object are predicted using the trained model generated in this way.

### <Operational Effects of Prediction Device 100 and Prediction System>

The prediction device 100 and the prediction system according to the present embodiment acquire the non-scientific information and the scientific information regarding the object and predict the plurality of characteristics of the object based on the acquired non-scientific information and the acquired scientific information. Thus, the plurality of characteristics of the object can be predicted concurrently. This operational effect will be described below.

As described above, promotion of DX has been desired in various fields. With DX, the number of manual work steps is reduced, and work efficiency is improved. However, there is a field for which sufficient DX has not been developed yet. For example, a plurality of characteristics such as mechanical properties and moldability of a product are often measured manually. In a case where a characteristic of a product is manually measured, there is a risk that the measured value varies due to an artificial factor.

In contrast, in the prediction system and the prediction device 100 according to the present embodiment, the plurality of characteristics of the object are predicted based on the non-scientific information and the scientific information regarding the object, and thus the plurality of characteristics of the object can be easily concurrently grasped. For example, it is possible to easily grasp the characteristics of the object throughout the manufacturing process and the life cycle, such as the tensile strength, impact strength, shape stability, and durability of the object. Therefore, it becomes easy to more efficiently obtain a socially valuable product while suppressing the number of manual work steps.

In particular, in the prediction system and the prediction device 100 according to the present embodiment, the prediction is performed based on the combination of the non-scientific information and the scientific information regarding the object, and thus it is possible to predict the plurality of characteristics of the object with higher accuracy. This will be described below.

The non-scientific information includes new multidimensional information that cannot be obtained from the raw data (scientific information) alone. Further, the scientific information includes information from which a phenomenon occurring in the object can be directly grasped and that is directly linked to a mechanism of a reaction or a mechanism by which a function is expressed. If the prediction is performed based on only the non-scientific information, information associated with the raw material of the object, the manufacturing process, and other phenomenon expressions are not considered, and thus it is difficult to grasp the effect of the quality (the amount of impurities or the like) of the raw material. On the other hand, if the prediction is performed based on only the scientific information, since structural information is not considered, for example, it is difficult to grasp a change in the strength of the plastic product (object) due to the orientation state of the fiber or the like.

Further, it is preferable to extract a feature of multidimensional information included in the non-scientific information by using deep learning. Thus, a pattern or a common point is found from an enormous amount of data, and the feature is simply extracted. Therefore, even in a case where a factor that affects a predetermined characteristic has not been clarified, that is, even in a case where a mechanism for expressing the predetermined characteristic has not been sufficiently understood, it is possible to extract a feature necessary for machine learning. This suggests the possibility that information that cannot be obtained from the scientific information or a feature that cannot be obtained from the scientific information can be obtained from the non-scientific information. In this way, by extracting the feature of the non-scientific information using deep learning, it is possible to predict the characteristics of the object with higher accuracy.

As described above, by acquiring both of the non-scientific information and the scientific information, it is possible to more accurately grasp characteristics such as the physical properties, quality, and functions of the object. That is, it becomes possible to predict the plurality of characteristics of the object with higher accuracy.

Operational effects of the prediction system and the prediction device 100 according to the present embodiment will be described in more detail.

The prediction system and the prediction device 100 according to the present embodiment are technologies related to a method such as inspection, detection, analytics, measurement, or sensing for manufacturing a wide variety of products in a small amount that are suitable for Society 5.0. For example, a state of a subject (substance), a subtle difference and a change in composition, and the like are inspected. The prediction system and the prediction device 100 according to the present embodiment relate to a device and a system that combine scientific information and non-scientific information as input-side data to obtain teacher data, and evaluate the teacher data by arithmetic operation using artificial intelligence and an algorithm. The scientific information is obtained from data that is obtained for the purpose of detecting material information, process conditions, minute differences and changes therein, or characteristics correlated therewith and for which a detection signal or information thereof is used or utilized as it is. The non-scientific information is obtained by processing data acquired for analyzing, analyzing, or evaluating the performance, function, quality, or the like of a certain object.

The prediction system and the prediction device 100 are related to various currently operated manufacturing industries and processing industries, and research and development, quality assurance, inspection, and analysis related to or accompanied by them, and are also intended to describe, record, and evaluate, with high sensitivity, a state of a substance related to a raw material or traceability or an ID in manufacturing.

### 1. For Application of Digital Transformation to Product Manufacturing

### 1-1. Issues in Manufacturing Industry and Changes Therein

In the manufacturing industry, which is also referred to as “product manufacturing”, most of acts of research and development, technical development, production, manufacturing, and the like are borne by subjective and tacitly intelligent activities or thoughts, which are referred to as “instincts”, “experience”, and “knacks” of engineers and craftsmen, and the fact that the transfer of their skills and know-how does not proceed as intended is highlighted as a social issue.

From such a background, factory automation has been adopted in various industries to contribute to improvement in productivity and quality. However, all of these are established in accordance with basic principles of economic activity of “mass consumption and mass production”, and is not consistent with a manufacturing industry which provides “a necessary thing in a necessary amount to a necessary person at a necessary time” defined by the Super Smart Society (Society 5.0) proposed by the Japanese government. In other words, the automation itself is a manufacturing method that should be denied in the Super Smart Society that is being developed and is expected to be materialized in 2030.

For example, 3D printers are means that can meet the demands of the above-described Super Smart Society for manufacturing three-dimensional objects, although they are applied to only a very limited number of manufacturing industries. However, materials that can be molded but are compatible with the 3D printers are only metals, alloys, and ceramics, and it is the actual situation that the most general-purpose plastics have hardly been applied on a commercial scale. Further, the 3D printers has various disadvantages. The disadvantages are that, due to the characteristics of the 3D printers, the mechanical strength of a shaped object varies depending on the orientation of the object, the manufacturing time is long, an unexpected large amount of wastes are generated, and there are many issues in terms of resource saving and SDGs.

### 1-2. Issues in Food Processing and Changes Therein

In addition to the so-called industrial products as listed above, as issues in the product manufacturing, for example, there are many issues in food processing.

For example, in response to the globalization of the manufacturing and distribution of food in the recent past, “mandatory introduction of HACCP” began on Jun. 1, 2020 also in Japan due to the revised Food Sanitation Law passed in June 2018. Thereafter, after a grace period of one year, “HACCP fully mandatory” has been required for all food-related companies from June 2021.

In HACCP, a manufacturing process is subdivided and risk management is performed for each step. Therefore, it is possible to prevent shipment of a product having a problem, and even if a food accident occurs, it is possible to quickly find out which step is the cause. However, since HACCP is a law required by more than just large-scale manufacturers, it is extremely difficult to manage all steps with advanced analytical equipment in terms of cost, and items to be managed are diversified, and therefore, handling it is a major issue.

In a conventional method, “sampling inspection” from “packaging” to “shipment” has been the mainstream. However, the HACCP method is a sanitary management method for ensuring the safety of a product by “predicting harm such as contamination by microorganisms or mixing of foreign substances” and “continuously monitoring and recording a particularly important step leading to prevention of harm” in each step from reception to processing/shipment of raw materials. Therefore, while it is possible to prevent shipment of more problematic products than the conventional sampling inspection of final products, costs and time and effort (man-hours) for inspection and analysis, furthermore, large-scale modification of manufacturing processes and the like have already become major problems.

Since HACCP is a system and regulation which have just started in Japan, it is not sufficiently understood by people other than those concerned in the industry that it is a major issue. However, it is obvious that it is indispensable to take measures against this issue from various angles beyond the food industry.

### 1-3. Issues in Quality Assurance

In the above-mentioned food processing and manufacturing, quality assurance is also an important act, and measures have been taken by various methods. However, what should be considered as a problem is that evaluation items of quality assurance are limited to practices and management of process conditions, and there are many cases where essential analysis on quality is not performed. The process conditions are, for example, heating at 100° C. for 2 minutes, or annealing at room temperature for 1 hour after shaping.

Further, for chemical products such as resin materials, representative physical properties such as elastic moduli and softening points are measured as specification items and listed in catalogs, quality certificates, and the like. For example, even if specification values are the same, the same characteristics are not necessarily obtained when products are processed. Therefore, it is also general that the user side secures the quality to a certain extent by his/her personal quality check relying on long-time experience and instincts and knacks of the person in charge.

Needless to say, such a quality assurance system is based on the premise of “mass production/mass consumption”, which have been the center of the manufacturing industry, and it is no longer possible to handle on-demand products for the Super Smart Society by sampling inspection. It is clear that a system for individually and simply maintaining and guaranteeing quality will be required in the future.

### 1-4. Issues in Inspection and Analysis

In a case where the style of consumption production, that is, the “common sense in the world” changes, it is naturally necessary to handle this change also in methods and means of inspection and analysis.

Up to now, the measurement accuracy has been improved more and more in machinery which can be purchased as an analyzer, the operability has been remarkably improved, and the size of the analyzer has been reduced, so that it is expected that the trend will not be largely changed in the future.

However, if the “common sense” of quality assurance and inspection related to manufactured products (including foods, pharmaceuticals, beverages, and the like) changes as described above, it should be considered ineligible to use such a conventional analyzer as a measurement tool for quality assurance in the Super Smart Society. In the future, new inspection and analysis methods will be required.

Here, consideration will be given to currently commercially available analyzers. An analyzer manufacturer naturally aims at increasing profits. That is, the analyzer manufacturer is not a charity. Therefore, the analyzer manufacturer markets only products which can be valued by many users. The most prolific users of analyzers are scientists who conduct academic research, typified by research departments of universities and companies. Further, many of the purposes for which such users use analyzers are to verify the logical nature of academic papers and dissertations. That is, logical support is always present in data generated from an analyzer, and the logical nature has no meaning unless at least a scientist on the user side can understand it.

On the other hand, whether or not all substances and foods subject to quality assurance can maintain their quality without scientific grounds is an issue. The present inventors have considered that it might be a fundamental problem. That is, in a case where there are a wide variety of objects that can be measured, simple measurement can be performed on those objects, a large amount of data can be generated, and the quality and characteristics can be guaranteed, even when the logical nature is not necessarily guaranteed, it may be sufficient for the purpose of “guaranteeing the quality”. The case where “the logical nature is not necessarily guaranteed” includes a case where although the logical nature is essentially present, the logical nature is not understood by humans.

The underlying problem-awareness of the present inventors was that there might be a necessary and sufficient inspection and analysis method for a manufacturing act in the Super Smart Society although it is not such a conventional analytical instrument and is not currently marketed.

### 1-5. Issues in Traceability

Although it also relates to the above-described inspection and analysis, when it comes to manufacturing “a necessary thing in a necessary amount to a necessary person at a necessary time”, traceability during the manufacturing should be rather important than the final characteristics and quality of the manufactured product. Producing a product with a strictly controlled raw material in a highly transparent manufacturing process leads to the quality, and it may lead to consumers' credit. If so, it is important to simply record and digitize the traceability of the manufacturing process.

1-6. Issues in IDs (State Records) of Raw Materials or Intermediate Processed Products for which Liability for Manufactured Products is Required

As on-demand manufactured products are widely distributed throughout the world, preparations for liability for the manufactured products become a major issue. The aforementioned traceability is also one of the preparations. For a raw material used, an intermediate product taken out during the manufacturing, and a final manufactured product, the identity and state of a substance of each of the raw material, the intermediate product, and the final manufactured product are recorded, that is, a wide range of identification is always required.

Even at present, many IDs are recorded using QR codes (registered trademark), barcodes, or the like. However, if IDs are required even for raw materials, it is essential to devise and spread a method by which IDs can be easily assigned and acquired even for people working in primary industries.

In addition, it is a major issue to store and manage IDs of all of raw materials, intermediate products, and final manufactured products in a case where they are further produced on-demand, and to trace back to the upstream side of manufacturing by connecting data when a problem or the like occurs. Current human-centered management cannot handle the issue, and it is premised that artificial intelligence (AI) is effectively utilized.

### 2. To Successfully Link Product Manufacturing, Data Science, and Computing Science

### 2-1. Current Issues in Transition Period to Data-Driven Research and Development

As a technology and research and development version of digital transformation, data-driven research and development such as bioinformatics and materials informatics have been spotlighted.
https://www.admat.or.jp/library/5975666db3de4b020a7803ae/61ea4ddafc46fdbc65f00a21.pdf

The international trend began with the Materials Genome Initiative initiated by the United States in 2011, and NOMAD in Europe, Creative Materials Discovery in Korea, and the like are performing activities as national projects so as to follow it. In Japanese as well, the initiatives that actively incorporate computational science and data-driven development are being implemented in research and development and technology development, centered on industry-government cooperation involving national research periods and private sectors, as key measures of the Ministry of Education, Culture, Sports, Science and Technology and the Ministry of Economy, Trade and Industry, such as the SIP innovative structural materials/MI system launched in 2014, the Mi2I in 2015 and later years, and the Ultra High-Throughput Design and Prototyping Technology for Ultra Advanced Materials Development Project (commonly known as Ultra-Ultra Pj) in 2016 and later years.
https://www.admat.or.jp/library/5975666db3de4b020a7803ae/61ea4ddafc46fdbc65f00a21.pdf

Among these, Ultra-Ultra Pj has completed the 6-year activity period, and for the 19 themes examined therein, the reduction rate of the development period or the number of trials has been specifically reported.
(https://www.admat.or.jp/library/5975666db3de4b020a7803ae/61ea4ddafc46fdbc65f00a21.pdf).

This project examined the benefits of combining calculations, processes, and measurements for the 19 themes. Therefore, the results are very helpful as a guide for future research and development and technical development.

Since the results reports on all the themes are uploaded on the web, when they are carefully read, the following overall image appears. No data-driven development was implemented from the beginning for any theme. Until data is accumulated, computational science seems to be mainly utilized for simulation.

In addition, in order to acquire a large amount of data, a high-throughput experimental apparatus or a high-throughput measurement apparatus is required because conventional experiments and analyses mainly performed by humans cannot handle the acquisition. These have been successful as a result of the progress of studies with industry-government-academia cooperation as a national project. However, it is expected that it is considerably difficult to carry out this in a single company or a laboratory of a university in terms of various skills, cost, and knowledge.

2-2. Issues when Data-Driven Type is Mainstream

“DX Report 2” (December 2020) of the Ministry of Economy, Trade and Industry contains the following information.

TABLE 5
Digital transformation
Digitalization of organization crossing/overall work/manufacturing process
Transformation of research and development and organization for
“value creation in accordance with market needs”
Digitalization
Digitalization of individual work and manufacturing processes
Digitization
Digitalization of analog and physical data

Although it can be interpreted in various ways, data-driven development corresponds to the transformation of research and development and organization for “value creation in accordance with market needs”, which is positioned as “digital transformation” in this figure.

On the other hand, in many manufacturing industries and laboratories of universities, digitalization of individual work and manufacturing processes, that is, even “digitalization” has not been achieved. Further, in reality, there are many cases where digitalization of analog and physical data, that is, “digitization” is not completed.

Of course, it is not impossible to skip digitization and digitalization and transfer directly to digital transformation (DX). However, in a case where the current method is used as a base, it is probably impossible to omit the two steps and transfer to DX, and some new tool or method is required.

### 2-3. Issues Regarding Logical Interpretation in General Manufacturing Industry

Although the initiatives that utilize image data in particular have been exemplified above, there is also another issue regarding data-driven development specific to the manufacturing industry.

One of the most effective data-driven technologies is currently e-commerce (electronic commerce). In this case, a large amount of specific data of consumers is required. This is so-called “big data”. It is fundamental to perform machine learning or deep learning on it by using AI, and perform recommendation (presentation of recommended candidates) for individual consumption behaviors. E-commerce has been developed in a completely different way from traditional distribution and wholesale industries. However, in this case, since a result of analyzing the big data is allowed with the same degree of accuracy as the recommendation, it is not necessary to give a logical explanation about the result.

On the other hand, data driving in the manufacturing industry and research and development is a method of deriving a solution in an inductive manner, and is not a deductive solution method backed by conventional theories and laws. Therefore, the data driving in the manufacturing industry and research and development is sometimes out of the common and unexpected, and there is an advantage of leading to new awareness. However, it is hardly acceptable in the present situation to directly use a result obtained from the data driving for the prescription of steps in the manufacturing industry as it is, and some logical consideration is necessarily required. This is the difficulty of the data driving in the manufacturing industry and research and development, and is also a hurdle that must be overcome.

### 2-4. Issues Regarding Data Acquisition and Data Management in Primary Industries

Further, in HACCP in food manufacturing described in the section 1-2, although the data driving will become the mainstream in this field in the future, data acquisition has been performed less in the primary industry than in the manufacturing industry (secondary industry). From this point of view as well, it is obvious that it will become a major issue in the whole society, and from this point of view as well, new simple and effective data acquisition means or a new simple and effective data acquisition method will be required.

### 2-5. Issues Regarding Forward-Problem Solver Type Computer Simulation

When the data-driven development is regarded as an inductive method, that is, as an inverse problem solving method, it can be said that a simulation for obtaining a solution by performing a high-speed operation using a computer in accordance with a scientific law or theory is a typical method of forward problem solving.

According to the result report material of Ultra-Ultra Pj described in 2-1., it does not seem that research and development periods in the data-driven development are shortened for all the themes, and a plurality of cases have been introduced in which only the forward problem solver type simulation has been fully used to lead to great results.

In consideration of results of these simulation technologies, in order to estimate a macro behavior of an object, molecular dynamics calculation (MD) is used from the first principles calculation of molecules and atoms in the nanometer or less to the submicron region. Further, it is understood that a “multi-scale simulation” that spans from nano through micron to millimeter scales is used as achievement means in a simulation technology such as application of a finite element method to a size of millimeter or greater.

Such a calculation requires a so-called supercomputer-class large ultrahigh-speed computing machine, and it is easily found that there seems to be a problem that it is difficult to perform the calculation except in a national project.

### 2-6. Issues Regarding Inverse Problem Solver Type Informatics

On the other hand, in inverse problem solver type materials informatics and process informatics, machine power (operation speed) as high as that of the advanced simulation is not required. Instead, a large amount of so-called “high-quality data” associated with a phenomenon or a substance is required.

Conventionally, high-quality data is generally acquired by the instrumental analysis described in 1-4., but in this case, the time and man-hours required for data acquisition are significant, and data acquisition itself becomes a rate-limiting factor in data driving.

In some themes of Ultra-Ultra Pj, many examples have been introduced in which computer simulation, which was originally utilized for logical understanding, was rationally used as data generation means for data driving, leading to great results. However, even in that case, a supercomputer is required, and the same issues associated with a computing machine as in 2-5. described above occur.

That is, data acquisition means for manufacturing industry or a data acquisition method for manufacturing industry is required, which is neither a virtual experiment, such as computer simulation, nor a real experiment with low productivity, such as conventional instrumental analysis.

2-7. Current Status of Development Method Using Computational Science from Both Directions

If most of data-driven data is generated by computer simulation, scientific consideration is possible because computer simulation is essentially a deductive method.

On the other hand, since a result by data driving is obtained by an inductive solution, basically, the reason and scientific basis thereof are unknown. However, since data itself is provided with a logical property, it is possible for a specialized scientist in a specific area to treat both of them, thereby making it possible to theorize, in a forward problem manner, a solution which has appeared in an inverse problem manner, and it is considered to be new rational development means in the manufacturing industry and research and development.

In addition to data acquired by virtual simulation, all of the above-described issues should be solved if there is data acquisition means that is different from conventional instrumental analysis but is associated with a substance and a phenomenon, is real, and has high productivity. Based on this basic idea, the present inventors have made a challenge to implement a new data acquisition system.

### 3. From Viewpoint of Current Initiatives at Product Manufacturing Site-Utilization of Image Data

### 3-1. Initiative to Utilize Image Data

As an initiative to support promotion of digital transformation (DX), a system for acquiring and utilizing image data to solve an issue has been published, which is an effective initiative also in the industry field.

Attention has been paid to the concept of the Internet of Things (IoT) in which various “things” such as various sensors and cameras are connected to the Internet and collected data is utilized. With the acceleration of DX, digital technology has become more prevalent in daily activities of companies and individuals than ever before. However, on the other hand, in the manufacturing industry, there are many production sites where the incorporation of digital technology and the promotion of DX have not been sufficiently implemented. The reason why the improvement using the digital technology in such a site has been delayed is that the initial introduction cost of the digital technology is high, it is difficult to secure personal resources familiar with the digital technology in recent years, and it is difficult to derive an optimal solution due to limited resources.

In contrast to such a current situation, in an initiative to solve the issue by acquiring and utilizing image data, various sensors and cameras that are relatively inexpensive are used, and once a system is built, many human resources familiar with the digital technology are not essential, and the system can be relatively easily introduced to a site of manufacturing industry or the like. Further, providing technologies and know-how in cooperation with a plurality of companies makes it possible to access a wide variety of resources, making it easier to obtain an optimal solution.

### 3-2. Example of Initiative to Utilize Image Data

An example of an initiative to utilize image data may include an “image IoT” system in which an image technology and an IoT technology are combined. With this system, it is possible to lead to proposal of a solution to a wide variety of issues and needs such as improvement in productivity and work safety.

Here, a generic name for a combination of technologies, which are a device mounting technology for collecting high-quality image data from a site (edge) utilizing a core technology, and an AI platform for integrating various sensor data to perform advanced recognition and determination, is defined as image IoT.

In recent years, the number of companies that utilize data and IT technologies and grow rapidly is increasing worldwide. These companies are growing worldwide by making full use of the latest software and cloud technologies and providing, as services, new value and experience to customers based on the use of data.

These businesses have created the concept of a platform in the process of growth, causing destructive innovation across industries and business categories. It is a well-known fact that it is essential to utilize such a platform in creating new businesses in future business.

An image IoT platform (IoT-PF) that utilizes image IoT technology to accelerate social DX with customers and partner companies has been proposed. The IoT-PF is a platform that provides not only on-site analysis processing execution and cooperation with clouds required by the edge IoT strategy but also a common function for responding to non-functional requirements such as device management and security required for actual installation of a device on site. By utilizing this, it is possible to efficiently and quickly provide a solution while focusing on user experience and the development of a differentiation function.

A common architecture for analyzing and utilizing camera video of a manufacturing site has been formulated by utilizing the IoT-PF. Since many of issues at a manufacturing site can be visualized by analyzing camera video, it has become possible to build functions such as “visualization of productivity in a manufacturing process” and “visualization of compliance with a labor safety rule” in a common system. It is considered that they can be easily developed for other applications in the future.

The IoT-PF is a collection of control technologies capable of acquiring on-site raw data and feeding back a result of analysis utilizing AI to the real world in real time in order to solve various issues of customers. Further, an ecosystem with a partner company is built and becomes a hub of customer value co-creation for providing the best service for the customer. It is expected to provide an optimal solution to various demands of “wanting to see” using image IoT technology.

### 3-3. Configuration of FORXAI (Registered Trademark) Image IoT Platform

This image IoT platform mainly includes the following constituent elements.

(1) “Imaging AI” that is a technology group of high-speed and high-precision AI learning/inference centered on images, such as AI library/accelerator, and an engine specialized in images; a high-speed and advanced AI processing technology group for performing image analysis. In particular, by using this image processing technology, there are strengths in three areas of “human behavior” such as posture estimation and human attribute detection, “advanced medical care” such as X-ray dynamic analysis and image biomarkers, and “inspection” such as defect detection and classification, and they are also future focus areas.

(2) “IoT Platform” that enables smooth data-processing/remote management/update between IoT devices and a cloud, (3) “Sensor device” that is a group of devices of own and other companies that are capable of processing information and exceed human visual abilities, such as MOBOTIX (registered trademark), LiDAR, and a gas leakage monitoring camera. A solution group is formed by combining these three integrated image IoT technologies with a technology of a partner company.

### Functional Configuration of System

The FORXAI IoT-PF includes three hierarchies of a cloud, an edge, and a device, and functions required for each of the cloud, the edge, and the device are prepared in advance.

### Cloud

Cloud services in the FORXAI IoT-PF have prepared APIs for executing management of data storage, search, and the like, transmission of emails and mobile push notifications, device management, and the like.

### Edge

The edge is a computer placed at a site and has a function of receiving information from the device, executing processing using deep learning or the like, and transmitting the result to the cloud.

### Device

The device refers to sensors and actuators installed at a site and a built-in system that controls the sensors and the actuators.

### System Implementable by IoT-PF

As an example of the solution, it is possible to acquire a moving image from a camera device on site, browse a result recognized by AI via the cloud, and notify, when a specific situation appears, a smartphone of it. In addition, the operating status of the device can be managed via the cloud.

### 3-4. Effects and Issues of Image IoT System

The above-described examples in which the image IoT system is utilized in the field of industrial manufacturing are mainly applied to the following field, and examples in which the image IoT system is used for enhancing explanatory variables in informatics (machine learning) in research and development are still limited. In this field, as reported in “Utilization of
FORXAI IoT Platform in Manufacturing Industry” (https://research.konicaminolta.com/jp/pdf/technology_report/2022/pdf/19_yoshizawa. pdf % 22), “Improvement of MFP Assembly Process by Skeleton Detection Algorithm of FORXAI Recognition”
(https://research.konicaminolta.com/jp/pdf/technology_report/2022/pdf/19_sonoyama. pdf % 22), and “Initiatives to Improve Sophistication of Gas Monitoring System for Smarting Industrial Security”
(https://research.konicaminolta.com/jp/pdf/technology_report/2021/pdf/18_asano. pdf), solutions for work efficiency improvement are provided by capturing human behavior. This field is production processes such as inspection and monitoring of products that provide solutions to characteristics such as performances, functions, qualities, and physical properties of objects to be manufactured (as indicated in https://linx.jp/product/mvtec/halcon/, for example).

Research and development have fundamentally supported the “product manufacturing” that is specific to Japan and has supported the industry of Japan so far, and the research and development are the place where the characteristics are realized. At the present time when utilization of AI and robotics has been generalized in conventional research and development methods and product manufacturing techniques, it goes without saying that significant transformation is required for the future. However, as described in the above Chapter 2, it is not rational to apply all of them to the data driven type development and the data driven type production, and it is also indispensable to utilize the conventional research and development and the computer simulation using a forward problem solving method for some of them.

In particular, scientific analysis represented by instrumental analysis is effective not only for supporting the consideration of researchers and engineers, but also as an objective variable when performing informatics. Therefore, it is important to understand the advantages and disadvantages of analysis, simulation, and informatics (machine learning) and then utilize them complementarily. In short, it is important to determine on a case-by-case basis whether or not it is necessary to understand “data driving” deductively, depending on the area to be applied or the development issue.

It is believed that by utilizing the image IoT system more widely in the industry field, the image IoT system will be advanced to produce products suitable for Society 5.0 along with the improvement in efficiency of an entire supply chain and the entire industry.

As described above, it has been indicated that image data is important, that not only inductive interpretation but also deductive interpretation are required, and that the development of data-driven technology should be applied to product manufacturing in product manufacturing in the industry area. However, under the present circumstances, it is considered that they are not sufficiently satisfied. It is considered that the reason is that, in the manufacturing industry also called “product manufacturing”, the actions of research and development, technical development, production, manufacturing, and the like are mostly borne by subjective and tacitly intelligent activities or thoughts, which are referred to as “instincts”, “knacks”, and “experience” of engineers and craftsmen, and the transfer of their skills and know-how does not proceed as intended. This is also highlighted as a social issue. Specific examples of these issues will be described.

### 1) Issues in Manufacturing of Plastic Products

At present, the first example of product manufacturing relying on “instincts”, “experience”, and “knacks” is the manufacturing of plastic products. In the first place, the reason why they rely on “instincts”, “experience”, and “knacks” is that they have had success in solving problems through the accumulation of their skills. However, from a different point of view, it is considered that there is no other index or data for determination that should be relied upon. Physical properties required for plastic products, such as strength, flexibility, and ease of post-processing, are determined based on various requirements, such as whether resin fiber is oriented, whether additives function, whether they are homogeneous in the products, and the surface states of the products. On the other hand, information of each of the physical properties is not necessarily easily grasped by visual evaluation or the like. The orientation of the fiber and the like are such that the state can be observed by an expensive analyzer or the like, and there is a problem that it is not easy to adapt it to a manufacturing site in terms of cost, time, and the like. That is, it can be said that there is an issue in the acquisition of a sufficient number of pieces of data that are necessary for both inductive interpretation and deductive interpretation and also necessary for data-driven technology development.

Here, when attention is paid to the manufacturing of a three-dimensional object, it is considered that the use of the above-described 3D printers are means which do not rely on tacit knowledge in order to set manufacturing conditions and the like as numerical values and data. However, materials that can be molded but are compatible with the 3D printers are metals, alloys, and ceramics, and it is the actual situation that the most general-purpose plastics have hardly been applied on a commercial scale. Further, due to the characteristics of the 3D printers, the 3D printers have various disadvantages such as the mechanical strength of a shaped object that varies depending on the orientation of the object, a long manufacturing time, an unexpected large amount of wastes, and many new problems in terms of resource saving and SDGs. In addition, in actual sites, the manufacturing and processing of plastics are mainly carried out by small and medium-sized companies, and most of steps involve human intervention, which can also be said to be a cause of difficulty in data acquisition.

### 2) Issues in Manufacturing of Rubber Products

General steps of manufacturing rubber include (1) a design step, (2) a refining step, (3) a vulcanizing and molding step, and (4) an inspection step. (1) In the design, material conditions such as amounts of a rubber raw material (raw rubber) and compounding agents (a plasticizer, a vulcanization accelerator, and the like) and process conditions such as processing time are determined so as to meet required performance. (2) In the roll, materials other than a crosslinking agent under the determined conditions are weighed, the compounding agents are added to the raw material rubber, and the mixture is kneaded to manufacture an unvulcanized rubber compound. (3) The vulcanizing and molding step is a step of vulcanizing (crosslinking) and molding the unvulcanized rubber compound manufactured by the refining into a product. The last (4) inspection is performed, and this inspection is usually performed not only in the last step but also in an intermediate step.

The number of compounding agents may exceed 10 depending on the required performance. Even if the mechanism and reactivity (ease of reaction) of a chemical reaction in the case of using one or several types of compounding agents at the same time can be understood, in a case where a plurality of compounding agents are mixed, the mechanism and reactivity affect each other, so that the mixing becomes complex. Therefore, it is very difficult to understand all of the mechanisms and reactivities of compounding agents, and it is easily conceivable that in most of these steps, they have to rely on “instincts”, “experience”, and “knacks”. That is, it can be said that the problem here is that, for a composite material in which a plurality of raw materials are mixed to cause a complex chemical reaction, it is very difficult to obtain all analysis data for understanding or grasping all phenomena, in other words, it is difficult to obtain the types and number of pieces of data necessary for explaining them. Further, similarly to the preceding section, most of steps in the manufacturing of rubber products involve humans, which makes it difficult to obtain data.

### 3) Issues in Food Processing

As an issue in food processing described in Chapter 1, all food-related companies are required to comply with the new regulation called “HACCP fully mandatory”. It can be said that it is an issue to provide means for acquiring types and the number of pieces of data necessary and sufficient for inspection and analysis that can be accepted not only by large-scale manufacturers but also by all people concerned in the industry, and a system that can ensure the quality of food by them.

Further, it is considered that this system is also related to an issue in Food Tech which can be said to be the same food processing but can be said to be a new industry. Food Tech is a new industry that combines food and technology and introduces IT technology into areas from the production of food materials to cooking processing, thereby creating added values for new food products, cooking methods, and the like that have not been available in the past. Specifically, Food Tech includes the spread of robots to the processing and manufacturing of foods as described above, and the research and development of food materials typified by stable manufacturing in plant factories and the manufacturing of substitute meats. In such research and development and manufacturing, it can be said to be an issue to provide a system for acquiring the types and number of pieces of data that are necessary and sufficient also in designing and stably producing better quality such as taste and texture as intended.

### 4) Issues in Manufacturing of Pharmaceuticals

Similarly to the above-mentioned food products, the manufacturing of pharmaceuticals is subject to strict regulations regarding control of manufacturing and quality. One of the most important standards is good manufacturing practice (GMP). GMP is a standard regarding manufacturing management and quality control of pharmaceuticals and summarizes requirements for manufacturing excellent pharmaceuticals having good quality, and the World Health Organization (WHO) decided its establishment in 1968, and in response to that, GMP is established in each country. GMP is defined such that products are safely manufactured and “constant quality” is maintained in all processes from arrival of raw materials to manufacturing and shipment of final products. Recently, in order to be consistent with the PIC/S GMP guideline which is the latest international standard, the GMP ministerial ordinance was revised for the first time in about 16 years, issued in March 2021, and enforced from Aug. 1, 2021.

Three principles of GMP are (1) “to minimize human errors”, (2) “to prevent contamination and quality degradation”, and (3) “to design a system that guarantees high quality”. This is a basic requirement for producing a product having the same high quality no matter who works at any time. Based on these three principles, by performing checking a plurality of times (double check) or taking a work record, human-mediated behaviors are managed and the number of mistakes in human behaviors is reduced due to identification display such as a product name and a lot number of a pharmaceutical. Also from this, it can be said that it has been recognized that human-mediated behaviors, raw materials, and conditions in a manufacturing process also affect the performance of the product, in this case, the pharmaceutical.

Further, even at present, as records other than behaviors of people, many IDs are recorded using QR codes (registered trademark), barcodes, or the like. However, if IDs are required even for raw materials, it is essential to devise and spread a method by which IDs can be easily assigned and acquired even for people working in primary industries.

Further, it is a major issue to store and manage IDs of all raw materials, intermediate products, and final manufactured products in a case where they are produced on demand, and to trace back to the upstream side of manufacturing by connecting data when a problem or the like occurs. Current human-centered management cannot handle the issue, and it is premised that artificial intelligence (AI) is effectively utilized.

That is, it can be said that it is an issue to provide means for acquiring the number and types of pieces of data that is necessary and sufficient for inspection and analysis that comply with regulations and are accepted by not only large-scale manufacturers but also all people concerned in the industry. As this data, information regarding artificial behavior, and information regarding the identity and state of a substance in each of raw materials to be used, an intermediate product in the case of being taken out during production, and the final manufactured product are necessarily required.

### 5) Issues in Supply Chain for Product Manufacturing

Although the issue in the incorporation of the data driven type technology development into the product manufacturing has been described, an issue from a different viewpoint is the implementation of platform-type DX integrating a supply chain. In a manufacturing site, it is rare that all of a process of producing a material and a raw material, a process of manufacturing a component, an assembly process, and sales are performed by one company. For example, in a supply chain in the automobile industry, a plurality of companies, such as a manufacturer that sells automobiles (has a brand of automobiles), a manufacturer of raw materials, a manufacturer that manufactures components from the raw materials, and a manufacturer that assembles the components, and sometimes universities and research institutions, have a relationship like a pyramid structure. Although DX is being promoted in companies, important information is treated as trade secrets among companies. In addition, as described in the preceding section, since it is not possible to share tacitly known information, the information is divided, and platform-type (integrated-type) DX is not established. This is not a major problem if a performance or characteristic to be designed is single or a simple product is to be manufactured. However, a major hurdle is present in the development of complex products, in other words, composite materials and complex materials in which a plurality of scientific phenomena occur simultaneously. On the other hand, platform-type DX is expected in various industries.

### 6) Issues in Trade-Off in Product Manufacturing

Products are manufactured at an actual site of product manufacturing, and most of the products are required to satisfy a plurality of functions and specification items. These items include not only raw materials, production, quality assurance, research and development, and manufacturing processes, but also aspects of the product life cycles such as storage stability and durability when the products are delivered to customers. In the issues described up to the preceding section, although not explicitly mentioned, the focus is on predicting the performance of one function or specification item and finding conditions that satisfy the performance, but in actuality, it is necessary to simultaneously satisfy a plurality of performances. Since some of the performances are in a trade-off relationship, it is required to design a plurality of performances at the same time.

The current issues listed so far will be summarized. In product manufacturing which will be demanded in the future, there is a demand for issue solving means which is suitable for compliance with SDGs and various regulations, not only for supply chains and their industries, but also for society as a whole. From the viewpoint of satisfying high performance and high functionality at the same time, it can be said that product manufacturing that will be required in the future is particularly complex and highly difficult manufacturing. In the design of an object, it is considered that these issues can be solved by not only means for acquiring data necessary and sufficient for inspection, analysis, and important determination but also an AI analysis system for integrating the data to concurrently predict a plurality of physical properties, functions, and quality of the object.

As means for acquiring necessary and sufficient data which is important here, it is considered to use both “non-scientific information” and “scientific information”.

As described above, it is an object to acquire the number and types of necessary and sufficient high-quality pieces of data in order to develop a data-driven technology. The non-scientific information and the scientific information are combined as means for implementing the necessary and sufficient number and types of the pieces of data, as described in the preceding section. Thus, it is considered possible to acquire an explanatory variable caused by a data type that is insufficient if only one of the two types of information is used. Further, with respect to the number of pieces of data, the fact that a plurality of explanatory variables can be acquired from multidimensional data such as non-scientific information can also be said that a large number of pieces of data are acquired as substantial man-hours. In addition, it is possible to further increase the number of pieces of non-scientific information in which methods such as image generation are combined, and it can be said that the issue can be solved.

As described above, the prediction system and the prediction device 100 according to the present embodiment acquire scientific information and non-scientific information regarding an object and predict a plurality of characteristics of the object based on the acquired scientific information and the acquired non-scientific information. A new data generation method and means therefor, which have been actually studied and found by the present inventors, will be described.

### Multidimensional Data

As described above, since the data-driven development is a destructive innovation in research and development, its key technology cannot be implemented by the conventional practice. In a case where a person analyzes data, two dimensions are most likely to be considered as dimensions of the data, and the data is basically three dimensional at most. This is also because coordinates can be assumed three-dimensionally, and the most important reason is that it is due to “orthogonality of the data”, and it is premised that no element of other data is included between data and data and no interference occurs.

On the other hand, in machine learning, orthogonality can be eliminated by an algorithm and an arithmetic operation, and therefore the number of dimensions can exceed 3 and can be, for example, 100 or 1000. In addition, in the case of a human being, it is not possible to obtain a solution when the solution is complex, but it is easy to perform weighting of explanatory variables necessary for regression by principal component analysis, LASSO analysis, or the like in informatics using machine learning, and thus there is no problem even in a case where the data has many dimensions.

Scientific information obtained from conventional instrumental analysis is basically independent scientific information in which orthogonality is guaranteed. Conversely, data in a virtual world using a computer can be acquired in multiple dimensions regardless of orthogonality, depending on how the data is acquired. However, basically, the quality of the data is low, and in order to improve it, it becomes necessary to perform high-precision and high-cost calculation with such a supercomputer as described above.

As one means for overcoming it, the present inventors have focused on an interaction between substances. The present inventors consider that, by acquiring data corresponding to a subtle change or difference in the interaction between the substances, it is possible to acquire high-quality data multi-dimensionalized and associated with to the substances themselves or the states of the substances.

### Satisfaction of Explanatory Variable (Data Type)

High quality associated with a state can be rephrased as acquiring an explanatory variable that can explain the entire state. In order to acquire the explanatory variable, a certain amount of data is required. Consider now a case where only scientific information is used. In this case, the scientific information includes much information directly relating to characteristics and quality of an object, and in research and development activities, since the activities are for the purpose of elucidation and understanding of a phenomenon, the amount of the scientific information is inevitably large. In contrast, non-scientific information is multi-dimensional information. It can be said that structural information which cannot be obtained only from raw data can be obtained from the non-scientific information, and an explanatory variable caused by the structure can be obtained only from the non-scientific information.

Further, data in which human behavior is recorded is also considered as non-scientific information. Behaviors of persons who perform various tasks in a manufacturing process include behaviors directly linked to scientific information such as the above-described processes of preparing and using raw materials and the process of inputting process conditions to a manufacturing apparatus. On the other hand, it is considered that information that is represented by so-called instincts, knacks, and experience and that a human being is not aware of or that is unrecognizable by a human being is included in behavior data, and the non-scientific information is grasped.

By utilizing the non-scientific information, it is possible to significantly increase the number of types of data and further the number of explanatory variables obtained therefrom without increasing the number of steps. It can be said that this is the only means for finding a solution for eliminating the trade-off which is an issue.

### Satisfaction of Data Amount

A sufficient amount of data is needed to perform machine learning. As one of the reasons why the number of pieces of data decreases, it is considered that, in the case of research and development or the development of elemental technologies, scientifically meaningful data and scientific information collected from so-called deductive considerations are used, which is also an essential cause. In this case, since non-scientific information such as an image includes various types of information as described in the satisfaction of the explanatory variable, as means for satisfying a data amount, in addition to a method of obtaining a large number of data from one image, a method of using image data generated using a machine learning method such as GAN or VAE from the viewpoint of increasing the number of pieces of data itself is also one method. An image generated by such a method can be referred to as non-scientific information.

### “Human Behavior” Recognition Technology

In the development of AI technology in the “human behavior” category, the development of algorithms for person detection, posture estimation, behavior recognition, and the like utilizing deep learning has been advanced. A robust “human behavior” recognition technology that learns a large number of site images so as not to perform erroneous recognition in any environment has been developed. In practice, both high recognition accuracy and high-speed processing have been achieved in 2D posture estimation, and the estimation is utilized in the following businesses.

Unlike posture estimation technologies on the premise that a person is imaged from the side with a camera, which have been widespread, a posture estimation method in which a posture can be recognized even from a ceiling camera has been developed for HitomeQ care support. A unique algorithm for estimating a region of a person and a posture of the person is utilized by using a positional relationship between human body parts such as the head and the lower legs as features.

The “human behavior” recognition technology by imaging with a ceiling camera is also utilized for analysis of a customer's stay time, behavior in front of a shelf, and the like in a “go insight” service in which data regarding a purchasing behavior process in a store is subjected to data analysis and linked to marketing activity.

As described above, the prediction device 100 and the prediction system according to the present embodiment can predict a plurality of characteristics of an object.

A modification example of the above-described embodiments will be described. A description of a similar configuration to that described in the above embodiments is not repeated.

### Modification Example

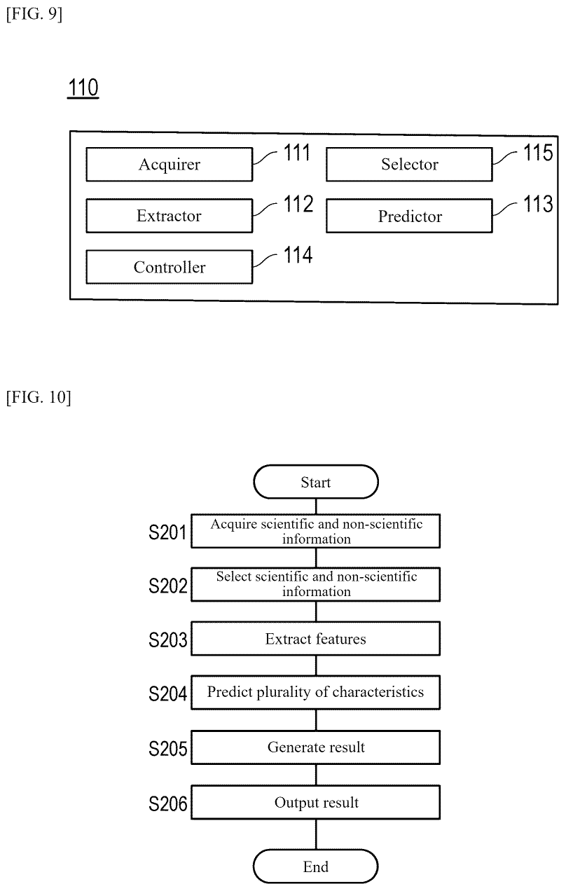

FIG. 9 illustrates a functional configuration of a prediction device 100 in a prediction system according to the modification example. The prediction device 100 may function as a selector 115 in addition to the acquirer 111, the extractor 112, the predictor 113, and the controller 114.

The selector 115 selects scientific information and non-scientific information in accordance with a plurality of characteristics of an object predicted by the predictor 113. The selector 115 selects, for example, scientific information and non-scientific information from among a plurality of pieces of scientific information regarding the object acquired by the acquirer 111 and a plurality of pieces of non-scientific information regarding the object acquired by the acquirer 111. The selector 115 may select a plurality of pieces of scientific information and a plurality of pieces of non-scientific information regarding the object. The selector 115 selects, for example, scientific information and non-scientific information highly relevant to each of the plurality of predicted characteristics.

The acquirer 111 may acquire the scientific information and the non-scientific information regarding the object selected by the selector 115. The selector 115 comprehensively selects, for example, the scientific information and the non-scientific information. For example, the scientific information is selected so as to include, as focal sizes of data, a plurality of sizes among a macro size, a micrometer size, and a nanometer size. Alternatively, the non-scientific information is selected so as to include, as structures of the object, a plurality of structures among a physical structure, a chemical structure, and an interface structure. The selector 115 may select scientific information and non-scientific information regarding the object using machine learning.

The predictor 113 predicts a plurality of characteristics of the object based on the scientific information and the non-scientific information selected by the selector 115. Thus, the accuracy of predicting each of the plurality of characteristics can be improved.

FIG. 10 is a flowchart illustrating a procedure of prediction processing executed in the prediction device 100.

### (Step S201)

First, the prediction device 100 acquires scientific information and non-scientific information regarding the object in the same manner as in step S101 described above in the embodiment. The prediction device 100 acquires, for example, a plurality of pieces of scientific information and a plurality of pieces of non-scientific information regarding the object.

### (Step S202)

Next, the prediction device 100 acquires scientific information and non-scientific information from the plurality of pieces of scientific information and the plurality of pieces of non-scientific information acquired in step S201, based on the plurality of characteristics to be predicted. The prediction device 100 may perform the processing in the order of step S202 and step S201.

### (Steps S203 to S206)

Thereafter, the prediction device 100 performs processing similar to steps S102 to S105 described above in the embodiment and ends the processing.

The prediction system and the prediction device 100 according to the modification example can also concurrently predict a plurality of characteristics of the object based on the scientific information and the non-scientific information regarding the object, similarly to the prediction system and the prediction device 100 described above in the embodiments. In addition, since the selector 115 is provided, it is possible to select scientific information and non-scientific information highly relevant to each of the plurality of characteristics to be predicted.

Therefore, it is possible to predict the plurality of characteristics of the object with higher accuracy.

### EXAMPLES

The effects of the present invention will be described using the following Examples. However, the technical scope of the present invention is not limited only to the following Examples. In the Examples, a fiber-reinforced resin in which a resin material and carbon fiber or glass fiber were mixed was used as a sample of the object.

### (Creation of Trained Discriminator)

First, in order to create teacher data, samples of 48 types of fiber composite materials were prepared. The samples were prepared by combining the following four types of resin, three types of fiber, fiber concentrations (volume ratios under two conditions, and injection pressure under two conditions. The resin and the fiber were mixed in advance at a desired ratio using a Labo Plastomill (registered trademark) extruder manufactured by Toyo Seiki Seisaku-sho, Ltd.

Thus, pellets were prepared. The samples of the 48 types of fiber composite materials were molded using an injection molding machine SE50D manufactured by Sumitomo Heavy Industries, Ltd. A sample shape for measuring a mechanical strength and a molding shrinkage rate was a dumbbell-shaped test piece type A1 shown in JIS K7139. A sample shape for measuring an impact strength was a test piece made by cutting the dumbbell-shaped test piece type A1 and forming a notch in a rectangular test piece indicated by JIS K7139B2.

The resin: polypropylene (Noblen (registered trademark) W101, manufactured by Sumitomo Chemical Co., Ltd.), polyamide 66 (Leona (registered trademark) 1300S, manufactured by Asahi Kasei Corporation), ABS (Toyolac 700 314, manufactured by Toray Industries, Inc.), polycarbonate (Iupilon (registered trademark) H-3000R, manufactured by Mitsubishi Engineering-Plastics Corporation); the fiber: PAN (polyacrylonitrile)-based carbon fiber (CF-N, manufactured by Nippon Polymer Sangyo Co., Ltd.), PAN-based carbon fiber (TC-3233, manufactured by Taiwan Plastics Corporation), glass fiber (CS3J-960, manufactured by Nitto Boseki Co., Ltd.); the fiber concentrations: 5% and 20%; and injection pressure: 50 MPa and 100 MPa.

Next, each of the samples of the 48 types of fiber composite materials was measured by using the following measurement devices, and features extracted from results of the measurement were learned by the discriminator. The measurement was performed near the center of the dumbbell-shaped test piece.

An FTIR (Fourier Transform Infrared Spectroscopy) device (AVATAR 370 manufactured by Thermo Fisher Scientific);
a terahertz-wave spectrometer (C12068-01 manufactured by Hamamatsu Photonics K.K);
an ultrasonic measurement device (UVM-2 manufactured by Ultrasonic Engineering Co., Ltd., measurement was performed in a reflection mode);
an X-ray diffraction device (Smart Lab manufactured by Rigaku Corporation);
an X-ray Talbot-Lau device (device described in Japanese Unexamined Patent Publication No. 2019-184450); and
a behavior moving image (moving image of a worker captured by a video camera).

The mechanical strength, the impact strength, and the molding shrinkage rate of each of the samples of the 48 types of composite resin materials were measured by the following methods, and the measurement results were learned by the discriminator.

Evaluation results of a tensile test performed using Tensilon (RTF-2325) manufactured by A&D Company, Limited in accordance with JIS K7161-2 were used as the results of measuring the mechanical strengths. In this case, a distance between grippers was 75 mm, and the test speed was 1 mm/minute. A value obtained by dividing the stress at break by the cross-sectional area of the test piece was defined as the mechanical strength. A Charpy impact test (U notch, R=1 mm) was performed in accordance with JIS-K7111, and evaluation results of the test were defined as the results of measuring the impact strengths. For the Charpy impact test, an impact tester (JCHBAS) manufactured by Toyo Seiki Seisaku-sho, Ltd. was used. The molding shrinkage rates were measured in accordance with JIS K7152-4.

### Examples 1 to 8 and Comparative Examples 1 and 2

First, samples of four types of objects were prepared. The samples were prepared by a combination of two types of resin, two types of fiber, a fiber concentration (volume ratio) under one condition, and injection pressure under one condition as indicated below. The samples were prepared in a manner similar to that of the teacher data.

The resin: polypropylene (Noblen W101 manufactured by Sumitomo Chemical Co., Ltd.), polyamide 66 (Leona 1300S manufactured by Asahi Kasei Corporation);
the fiber: PAN-based carbon fiber (CF-N manufactured by Nippon Polymer Sangyo Co., Ltd.), PAN-based carbon fiber (TC-33 manufactured by Taiwan Plastics Corporation);
the fiber concentration: 10%; and
the injection pressure: 80 MPa.

In Examples 1 to 8, scientific information and non-scientific information listed in Table 1 below were generated for the samples of the four types of objects. Thereafter, features extracted from the scientific information and the non-scientific information were input to the trained discriminator, and predicted values of the mechanical strengths, the impact strengths, and the molding shrinkage rates were obtained. In Comparative Example 1, only scientific information was generated, and in Comparative Example 2, only non-scientific information was generated. Thereafter, the scientific information or the non-scientific information was input to the trained discriminator, and predicted values of the mechanical strengths, the impact strengths, and the molding shrinkage rates were obtained.

Further, by using the same method as the creation of the trained discriminator, the mechanical strength, the impact strength, and the molding shrinkage rate of each of the samples of the four types of objects were measured, and the measured values were obtained. Next, errors between the predicted values and the measured values were calculated using the following Formula (1), and then the average of the errors of the samples of the four types of objects was obtained. In Table 1 below, a case where the average value of the errors is equal to or less than 30% is described as A, a case where the average value is greater than 30% and equal to or less than 60% is described as B, and a case where the average value is greater than 60% is described as C. That is, when the mechanical strength, the impact strength, or the molding shrinkage rate is “A”, it indicates that the accuracy of the characteristics predicted using the trained discriminator is the highest.

$$\begin{matrix}[ Equation 1 ] & \\ Error ( \% ) = \vert predicted value - measured value ) / measured value \vert \times 100 & ( 1 )\end{matrix}$$

TABLE 1

| | | | | | Molding |
|---|---|---|---|---|---|
| | | Non-scientific | Mechanical | Impact | shrinkage |
| | Scientific information | information | strength | strength | rate |
| Example 1 | Type and amount of contained substance, | Talbot image | A | B | B |
| | IR spectrum | | | | |
| Example 2 | Type and amount of contained substance, | Ultrasound image | A | A | B |
| | IR spectrum | | | | |
| Example 3 | Type and amount of contained substance, | Talbot image | A | A | B |
| | terahertz spectrum | | | | |
| Example 4 | Type and amount of contained substance, | Talbot image, | A | A | A |
| | IR spectrum | moving image of | | | |
| | | human behavior | | | |
| Example 5 | XRD spectrum | Talbot image | B | B | B |
| Example 6 | XRD spectrum, | Talbot image | A | B | A |
| | IR spectrum | | | | |
| Example 7 | IR spectrum | Talbot image, | B | A | B |
| | | ultrasound image | | | |
| Example 8 | XRD spectrum, | Talbot image, | A | A | A |
| | IR spectrum | ultrasound image | | | |
| Comparative | Type and amount of contained substance, | — | B | B | C |
| Example 1 | IR spectrum | | | | |
| Comparative | — | Talbot image | C | C | C |
| Example 2 | | | | | |

The errors were smaller in Examples 1 to 8 in which the features of the scientific information and the non-scientific information were input to the discriminator than in Comparative Examples 1 and 2. In addition, among Examples 1 to 8, in Examples 4, 7, and 8 in which a plurality of pieces of non-scientific information regarding the object were used, the errors could be reduced as compared to the other Examples.

The configurations of the prediction device 100 and the prediction system described above are merely main configurations for explanation of the features of the above-described embodiments and Examples, and are not limited to the above-described configurations and can be variously modified within the scope of the claims. In addition, a configuration included in a general prediction system is not excluded.

For example, the prediction device 100 may include constituent elements other than the above-described constituent elements, or may not include some of the above-described constituent elements.

Further, each of the prediction device 100, the first device 200, and the second device 300 may be configured by a plurality of devices or may be configured by a single device.

Further, the functions of each component may be implemented by another component. For example, the first device 200 or the second device 300 may be integrated into the prediction device 100, and some or all of the functions of the first device 200 and the second device 300 may be implemented by the prediction device 100.

Also, the processing units of the flowcharts in the above embodiments are divided according to the main processing contents in order to facilitate understanding of each process. The present invention is not limited by how the processing steps are classified. Each process can be further divided into more processing steps. In addition, one processing step may execute more processing.

The means and methods for performing various types of processing in the system according to the above-described embodiment can be implemented by any of a dedicated hardware circuit and a programmed computer. The program may be provided, for example, by a computer-readable recording medium such as a flexible disk and a CD-ROM, or may be provided online via a network such as the Internet. In this case, the program recorded on the computer-readable recording medium is usually transferred to and stored in a storage section such as a hard disk. Further, the program may be provided as a single piece of application software.

The program may be incorporated in software of the device as a function of the system.

The present application is based on Japanese Patent Application No. 2022-105570 filed on Jun. 30, 2022, the disclosure content of which is incorporated by reference in its entirety.

### REFERENCE SIGNS LIST

100 prediction device
110 CPU
111 acquirer
112 extractor
113 predictor
114 controller
115 selector
120 ROM
130 RAM
140 storage
150 communication interface
160 display
170 operation acceptance section
200 first device
300 second device

1. A prediction device comprising a hardware processor that:
acquires first information including an image regarding an object and second information including at least one of a character, a number, a chemical structure, and a spectrum regarding the object; and
predicts a plurality of characteristics of the object based on the acquired first information and the acquired second information.

2. The prediction device according to claim 1, wherein the hardware processor:
selects the first information and the second information in accordance with the plurality of characteristics of the object to be predicted, and
predicts the plurality of characteristics of the object based on the selected first information and the selected second information.

3. The prediction device according to claim 1, wherein the image includes an image obtained by imaging the object using at least one of an imaging device, an X-ray Talbot-Lau device, an ultrasonic device, a fluorescent fingerprint measurement device, a hyperspectral camera, a millimeter wave imaging device, a scanning electron microscope, an atomic force microscope, a transmission electron microscope, a fluorescence microscope, and a multidimensional colorimeter.

4. The prediction device according to claim 1, wherein the image includes an image obtained by imaging a behavior of a person related to the object.

5. The prediction device according to claim 1, wherein the second information includes at least one of a character and a chemical structure representing a type of a substance contained in the object, and a number representing an amount of the substance contained in the object.

6. The prediction device according to claim 1, wherein the second information includes at least one of an infrared absorption spectrum, a terahertz wave spectroscopy spectrum, a nuclear magnetic resonance spectrum, a Raman spectroscopy spectrum, an impedance spectroscopy spectrum, and an X-ray diffraction spectrum of the object.

7. The prediction device according to claim 1, wherein the object is a mixture of a plurality of substances having chemical structures different from each other.

8. The prediction device according to claim 1, wherein the plurality of characteristics include at least one of a physical property, quality, and a function of the object.

9. The prediction device according to claim 1, wherein the plurality of characteristics include at least one of a mechanical property, a physical property, a thermal characteristic, moldability, an electrical characteristic, durability, machinability, and combustibility of the object.

10. The prediction device according to claim 1, wherein the hardware processor causes an output section to output information regarding the plurality of predicted characteristics.

11. The prediction device according to claim 1, wherein the hardware processor predicts the plurality of characteristics using a trained discriminator.

12. The prediction device according to claim 11, wherein the hardware processor extracts a feature from each of the acquired first information and the acquired second information, and predicts the plurality of characteristics with the extracted features as inputs.

13. The prediction device according to claim 12, wherein the discriminator is subjected to machine learning with the features as input data and the plurality of characteristics as output data.

14. A prediction system comprising:
a first device that generates first information regarding an object;
a second device that generates second information regarding the object; and
the prediction device according to claim 1.

15. A non-transitory recording medium storing a computer readable prediction program for causing a computer to execute a process comprising:
acquiring first information including an image regarding an object and second information including at least one of a character, a number, a chemical structure, and a spectrum regarding the object; and
predicting a plurality of characteristics of the object based on the acquired first information and the acquired second information.
